(19) United States
(12) Patent Application Publication
(10) Pub. No.: US 2018/0095152 A1
(43) Pub. Date: Apr. 5, 2018

(54) SYSTEM AND METHOD FOR ESTIMATING A PHYSIOLOGICAL PARAMETER OF AN ELEMENTARY VOLUME

(71) Applicant: OLEA MEDICAL, La Ciotat (FR)

(72) Inventors: Bruno TRIAIRE, Les Pennes Mirabeaux (FR); Timothé BOUTELIER, La Ciotat (FR)

(73) Assignee: OLEA MEDICAL, La Ciotat (FR)

(21) Appl. No.: 15/560,647

(22) PCT Filed: Mar. 22, 2016
(86) PCT No.: PCT/FR2016/050626

§ 371 Date: Sep. 22, 2017

Related U.S. Application Data
(60) Provisional application No. 62/136,931, filed on Mar. 23, 2015.

(30) Foreign Application Priority Data
Jun. 19, 2015 (FR) 1555656

(51) Int. Cl. G01R 33/50; G01R 33/48; G01R 33/56; G01R 33/565; A61B 5/055

(52) U.S. Cl. CPC G01R 33/50; G01R 33/4816; A61B 5/055; G01R 33/565; G01R 33/5608

(57) ABSTRACT

A system and a method for producing an estimation of a physiological parameter of an elementary volume, termed a voxel, of an organ is implemented by a processing unit of a magnetic resonance imaging analysis system. The method includes a step for estimating the physiological parameter, the step for estimating the physiological parameter including producing the estimated value of the physiological parameter on the basis of the respective prior estimations of first and second physiological parameters.

This invention relates to a system and method for estimating a physiological parameter from data resulting from the acquisition of medical images. The invention is distinguished in particular from known methods by its accuracy and its speed of execution.

The invention is based in particular on Magnetic Resonance Imaging techniques (also known by the abbreviation “MRI”). These techniques allow valuable information about the organs of human beings or animals to be obtained quickly. This information is particularly crucial for a practitioner seeking to establish a diagnosis and to make a therapeutic decision in the treatment of pathologies.

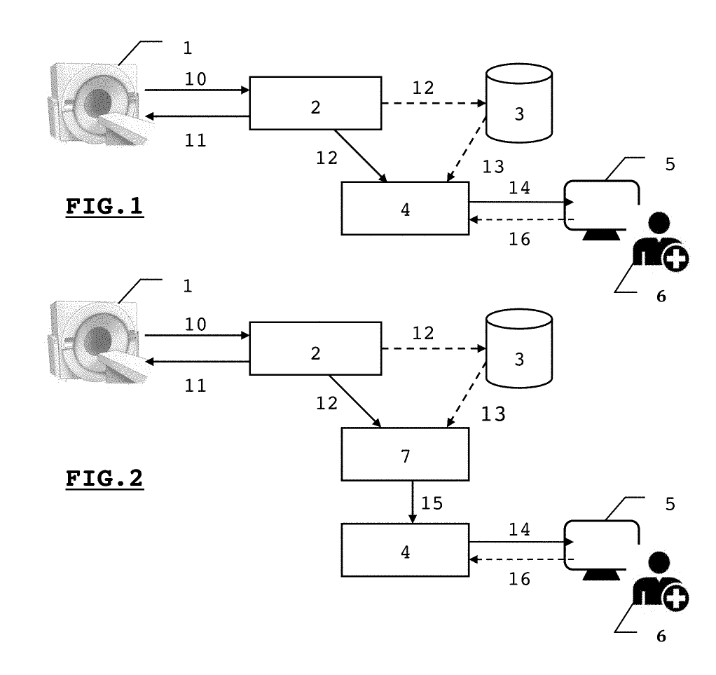

In order to perform these techniques, a Nuclear Magnetic Resonance Imaging device 1, as shown by way of non-limiting example in FIGS. 1 and 2, is generally used. It can deliver a plurality of sequences of digital images 12 of one or more parts of a patient's body, such as, by way of non-limiting example, the brain, the heart and the lungs. For this, said device applies a combination of high-frequency electromagnetic waves to the body part in question and measures the signal re-emitted by certain atoms such as, by way of non-limiting example, hydrogen, for Nuclear Magnetic Resonance Imaging. The device thus enables the chemical composition and therefore the nature of the biological tissues in each elementary volume, commonly termed a voxel, of the imaged volume to be determined. The Nuclear Magnetic Resonance Imaging device 1 is controlled by means of a console 2. A user can thus choose parameters 11 to control the device 1. From information 10 produced by said device 1, a plurality of sequences of digital images 12 of a body part of a human or animal is obtained.

The sequences of images 12 can optionally be stored in a server 3 and constitute a medical file 13 of a patient. Such a file 13 can comprise images of different types, such as perfusion or diffusion images. The sequences of images 12 are analyzed by means of a dedicated processing unit 4. Said processing unit 4 comprises means to communicate with the outside world in order to collect the images. Said communication means also allow the processing unit 4 ultimately to deliver, through output means 5 outputting a graphic, sound or other rendering, to a user 6 of the analysis system, in particular a practitioner or a researcher, an estimation of one or more physiological parameters, possibly formatted in the form of a content, on the basis of images 12 obtained by Magnetic Resonance Imaging by means of an appropriate man/machine interface. Throughout the document, “output means” refers to any device on its own or in combination that enables a representation, for example a graphic, sound or other representation of an estimated physiological parameter to be output to the user 6 of a Magnetic Resonance Imaging analysis system. Such output means 5 can consist non-exhaustively in one or more screens, loud speakers or other man/machine interfaces. Said user 6, advantageously a practitioner, of the analysis system can thus confirm or contradict a diagnosis, decide upon a therapeutic action that he deems appropriate, explore research work in greater depth, etc. Optionally, this user 6 can parameterize the operation of the processing unit 4 or output means 5 by means of parameters 16. For example, he can thus define display thresholds or choose the estimated parameters for which he wishes to have, for example, a graphic representation. There is a variation, described in relation to FIG. 2, whereby an imaging system, as previously described, also comprises a pre-processing unit 7 to analyze the sequences of images, deduce experimental signals 15 therefrom and deliver the latter to the processing unit 4, which is thus relieved of that task. Moreover, in order to make an estimation of physiological parameters, the processing unit 4 usually comprises processing means, such as a computer, to implement an estimation method in the form of a program preloaded into the storage means cooperating with said processing means. More generally, the processing unit can consist in one or more microprocessors or microcontrollers and/or internal memories cooperating with said microprocessors or microcontrollers. The notion of a processing unit can also extend to any operating system software resource, implemented by said hardware elements, that offers services to facilitate the management of the hardware resources of said processing unit for any application method implemented by the latter.

Thus, the acquisition of data, advantageously signals, by Magnetic Resonance Imaging, henceforth referred to as MRI, can be performed by regularly sampling a parallelepiped volume along a given slice plane. The two-dimensional images obtained consist of pixels of a thickness corresponding to the slice thickness and called voxels.

For any given voxel, the signal S obtained with the aid of said MRI acquisition system depends on two types of parameters.

On the one hand, such a signal S depends on physiological parameters, namely the magnetic properties of the tissue that are, for example:
the longitudinal relaxation time T1 (spin-lattice): the longitudinal relaxation corresponds to the process bringing magnetization to equilibrium according to the direction of the magnetic field B₀. T1 is the characteristic time for establishing magnetization when the sample is placed in the magnetic field or that which characterizes the return to equilibrium after an inversion. T1 is also the interval of time corresponding to the recovery of 63% of the initial longitudinal magnetization;
the transverse relaxation time T2 (spin-spin): transverse relaxation is the process of return to equilibrium, namely to zero, of a magnetization brought into the plane perpendicular to the magnetic field B₀. This magnetization decreases with a characteristic time T2. T2 is the interval of time corresponding to the loss of 63% of the initial transverse magnetization since ceasing the application of a radiofrequency;
the PD (Proton Density).
On the other hand, said signal S depends on acquisition parameters directly linked to the Nuclear Magnetic Resonance Imaging device 1, said parameters thus being applicable to all the voxels. These acquisition parameters are for example:
the echo time TE: interval of time between an excitation by means of a pulse and the occurrence of an MRI signal in response to said excitation;
the repetition time TI: interval of time between two excitations;
the flip angle α;
the inversion time TI: interval of time between two characteristic pulses of a sequence for specific acquisitions in the context of inversion-recovery MRI.

By way of a non-limiting example, according to a first embodiment, during an acquisition sequence called a spin echo, the signal S can be defined according to the following proportionality relation:

S ∝ PD[1−e^{TR/T1}]e^{-TE/T2}

By cleverly and manually combining the acquisition parameters by means of a prior configuration of a Nuclear Magnetic Resonance imaging device, a user of the device is able to obtain weighted images or image sequences in T1, T2, PD, or even of obscuring certain types of tissues. Thus, the user 6 can influence the generation of images. When the user chooses for example a low value of TR, the term dependent on T1 can then be ignored and ultimately the signal S is solely and substantially dependent on the physiological parameter T2. When an image or map is then generated, said image is described as a T2 weighted image.

According to a second embodiment, during an inversion-recovery acquisition sequence in spin echo, the signal S can be defined according to the following proportionality relation:

S ∝ PD[1−2e^{T1/T1}+e^{TR/T1}]e^{T2/TE}

According to another variation, like the first embodiment, if the user chooses the acquisition parameter T1 appropriately, said user can then generate images lacking or omitting a certain type of tissue, by way of non-limiting examples fat or tissues.

According to a third embodiment, during a gradient echo acquisition sequence, the transverse relaxation time T2 is modified by magnetic field heterogeneity effects. In fact, the magnetic field applied within the imaging device is not perfect since the magnet inducing the magnetic field is not uniform. The transverse relaxation time is then called T2*. The signal S can then be defined according to the following proportionality relation:

$$S ∝ PD \frac{sin α [ 1 - {e}^{- TR / T 1} ]}{[ 1 - cos α \cdot {e}^{- TR / T 1} ]} {e}^{- T 2 / TE}$$

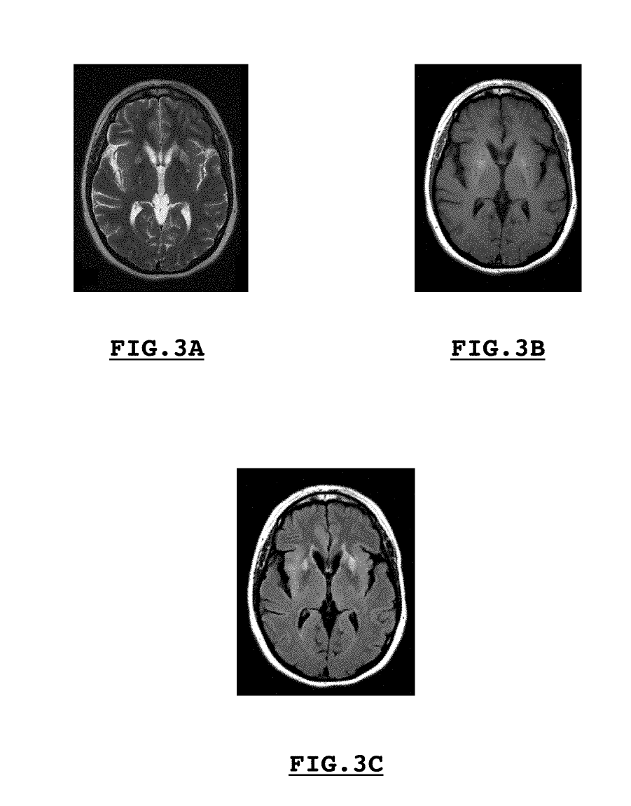

FIGS. 3A, 3B and 3C show three examples of maps or images that are T1 weighted, T2 weighted and inversion-recovery respectively, obtained by a Nuclear Magnetic Resonance device according to a choice of parameters defined by a user. These Figures show different data contrasts, allowing certain parts of the brain to be highlighted. According to FIG. 3C, for the inversion-recovery sequence, the user has chosen adjustment of acquisition parameters TR, TE and TI so as to suppress the signal induced by water.

Depending on the embodiment, in other words the chosen acquisition sequence and the adjustment of the acquisition parameter, the user, by means of an appropriate imaging analysis system, can generate different types of weighted images which, as stated above, enable different organs of interest to be highlighted. Thus, a user, advantageously a practitioner, can then use these weighted images to establish a diagnosis, for example the location and/or characterization of a tumor.

In most cases, in order to enable the establishment of said diagnosis, the user or practitioner must perform several acquisition sequences in order to obtain different types of weighted images and consequently different contrasts. The acquisition sequences are usually quite long, in the order of several minutes. The multiplication of sequences consequently considerably increases the duration of the examination and causes several negative consequences such as, but not limited to:
discomfort for the patient, said patient having to stay still for a long period of time in an anxiety-invoking and stressful environment;
a low frequency of examination of patients as the examinations are of relatively long duration, resulting in waiting lists that are sometimes extremely long for a patient requiring an MRI examination;
a high examination cost, said cost being notably proportional to the acquisition time.

In order to overcome these drawbacks, some researchers have devised methods or processes, advantageously implemented by a processing unit of a Magnetic Resonance Imaging analysis system, consisting globally in estimating physiological parameters, such as parameters T1, T2, T2*, or PD. According to these methods, after having estimated said parameters T1, T2, T2*, or PD. by means of appropriate methods, it is possible to generate artificially and manually, i.e. in a manner that cannot be easily reproduced and is tedious, a weighted image or contrast using an equation linking the intensity of the signal at the parameters of the tissue to those of the desired sequence. This is referred to as Synthetic Magnetic Resonance Imaging.

According to a first example of application, methods, advantageously implemented by a processing unit of a Magnetic Resonance Imaging analysis system, have been elaborated in order firstly to acquire images from multi echo spin echo sequences, then calculate and/or estimate the physiological parameter T2 (methods also known as “T2 mapping sequences”) and then generate weighted images, whatever the value of the acquisition parameter TE. The creation of weighted images is however limited to the creation of synthetic T2-weighted maps, since the acquisition parameter TR cannot vary.

According to a second example of application, similar methods, also implemented by a processing unit of a Magnetic Resonance Imaging analysis system, can be applied to estimate and/or calculate the physiological parameter T1, by advantageously varying the parameter TR or the flip angle a (methods also known as “ T1 mapping sequences”). In the same way as before, by advantageously varying the parameter TR or the flip angle a, these methods can allow weighted images to be generated, also known as “maps”, while keeping the acquisition parameter TE constant. These maps are then qualified solely by T1-weighted images.

According to a third example of application, by using a multi-echo gradient echo sequence, similar methods can be applied to estimate and/or calculate the physiological parameter, parameter T2* (methods also known as “T2*mapping sequences”). In the same way as before, by advantageously varying the parameter TE only, such methods can enable weighted images to be generated, while keeping the acquisition parameter TR constant. Such images or maps are then qualified by T2* weighted images.

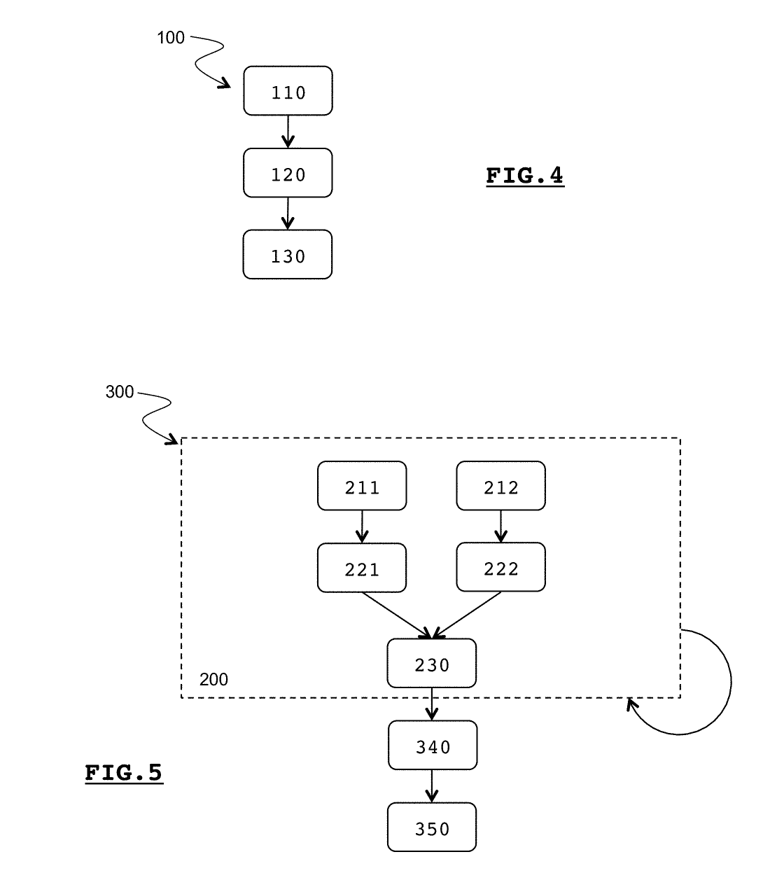

Alternatively or additionally, other researchers have devised a method, proposing a special acquisition protocol enabling, by means of a single sequence (also known as “QRAPMASTER”), the estimation of the physiological parameters T1, T2 and PD simultaneously. This method 100 is described in relation to FIG. 4. Said method 100 comprises a series of three successive steps:
a first step 110 to acquire experimental signals by means of the single QRAPMASTER sequence;
a second step 120 to estimate simultaneously the physiological parameters T1, T2 and PD;
a third step 130 to generate any type of image of spin-echo or inversion-recovery sequences.

The fact that the methods and processes described above enable any type of images to be generated at will and instantaneously from one or more acquisition sequences offers a certain number of advantages. Firstly, these methods enable the duration of examinations to be shortened and, consequently, their costs to be reduced and patient comfort to be improved. Furthermore, synthetic MRI maps or images, obtained with the aid of the methods described above, are free from noise, aside from that generated by uncertainty as regards measurements T1, T2 and PD, etc. These maps or images are thus of excellent visual quality. Thanks to the methods and images obtained, a practitioner can then anticipate the adjustments that will enable him to obtain the desired contrasts before making a new acquisition, a real one this time.

Among the existing methods, the most efficient is the method based on the QRAPMASTER sequence. In fact, this method enables the estimation, in just one acquisition, of all the relevant physiological parameters necessary for the generation of images, particularly spin echo or inversion-recovery images. However, this method has a certain number of drawbacks. In fact, such a method relating to this type of sequence is very specific and cannot be applied to all Magnetic Resonance Imaging analysis systems. Consequently, such a method has very high implementation and maintenance costs for the establishment that wishes to use it, in the order of hundreds of thousands of dollars. Moreover, such a method is applicable only to one type of organ, the brain. The other organs cannot therefore be analyzed.

Other methods or processes enable weighted synthetic maps to be generated for one physiological parameter, namely either T1 or T2. In fact, these methods involve a step of estimating a physiological parameter, either T1 or T2 respectively. The practitioner must study several maps or weighted images simultaneously in order to obtain all of the physiological parameters, without however being able to recover directly all of the information in one and the same weighted image. The practitioner's task is therefore not easy; indeed, it is laborious.

Moreover, the step of estimating parameters T1, T2 or PD usually consists in a step of calculation by linearization of the equations linking the physiological parameters to the signal S. The calculation times are certainly reduced. However, said estimations are very sensitive to noise. These methods thus dictate the use of sequences for which the signal-to-noise ratio (SNR) is high, risking obtaining maps or weighted images that are unreliable or incorrect. In order to obtain satisfactory signal-to-noise ratios, it becomes necessary to increase the acquisition times. Thus, the increase in acquisition times, considerably increasing the duration of the examination, has the same negative consequences referred to above, such as discomfort for the patient, low frequency of examination of patients due to the relatively long duration of examinations, and a high examination cost.

The invention resolves the vast majority of the drawbacks raised by known solutions.

Among the many benefits of the invention, we can mention that it allows:
proposing an economical solution, applicable to all common or conventional Nuclear Magnetic Resonance Imaging analysis systems;
reducing the duration of examinations, by reducing the number of acquisition sequences;
enabling it to be used to analyze any organ or type of organ, indeed a patient's entire body;
obtaining better results by considerably improving noise robustness, thus enabling a considerable reduction in acquisition times for the sequences required to implement the invention;
improving the characterization of the tissues and segmentation of said tissues.

To this end, a method is notably provided in order to produce an estimation of a physiological parameter of an elementary volume—termed a voxel—of an organ, said method being implemented by processing means of a processing unit of a Magnetic Resonance Imaging analysis system, and comprising a step for estimating said physiological parameter. According to the invention, such a method comprises a step for estimating a first physiological parameter on the basis of the first experimental signals resulting from a first acquisition of signals, as well as a step for estimating a second physiological parameter on the basis of second experimental signals resulting from a second acquisition of signals. Furthermore, said step for estimating the physiological parameter involves producing the estimated value of said physiological parameter on the basis of respective estimations of the first and second physiological parameters.

To enable rapid and particularly effective diagnoses, a method according to the invention may also comprise steps to produce the first and second experimental signals respectively on the basis of first and second acquisitions of signals.

According to a preferred embodiment, the invention envisages that the step to estimate a first physiological parameter can consist in a step of estimation by a Bayesian method, involving estimating said first physiological parameter by calculating its marginalized posterior distribution.

Similarly, preferably, the invention envisages that the step to estimate a second physiological parameter consists in a step of estimation by means of a Bayesian method, involving estimating said second physiological parameter by calculating its marginalized posterior distribution.

Advantageously, when the Magnetic Resonance Imaging analysis system comprises means of output to a user of said system, said output means cooperating with the processing unit, a method according to the invention can comprise a subsequent step for triggering an output of the estimated physiological parameter and/or of the first and second physiological parameters.

To improve the quality of the experimental signals obtained and acquired by Magnetic Resonance, a method according to the invention can also comprise a prior step of pre-processing the first and/or second experimental signals obtained from the first and/or second acquisitions by Magnetic Resonance respectively, said step being arranged to correct the said first and/or second experimental signals.

According to a second subject-matter, the invention concerns a method for producing an estimation of a physiological parameter of a region of interest, said region comprising at least one voxel. According to the invention, said physiological parameter is estimated for each voxel by means of a method according to the first subject-matter of the invention.

Advantageously, when the Magnetic Resonance Imaging analysis system comprises means of output to a user of said system, said output means cooperating with the processing unit, a method according to the invention can also comprise a subsequent step to trigger the output and estimation of the physiological parameter, of the first and/or second physiological parameters for each voxel of the region of interest in the form of a map describing a physiological parameter.

In addition or as a variation, when the Magnetic Resonance Imaging analysis system comprises means of output to a user of said system, said output means cooperating with the processing unit, a method according to the invention can also comprise a subsequent step to generate a weighted image on the basis of the values produced from the estimated physiological parameter, the first and second physiological parameters for a predetermined acquisition sequence.

According to a third subject-matter, the invention concerns a processing unit comprising means to communicate with the outside world and processing means, cooperating with storage means. Advantageously, the communication means are arranged to receive from the outside world first and second experimental signals based on the first and/or second acquisitions of signals by Magnetic Resonance and the storage means contain instructions that can be executed or interpreted by the processing means, the interpretation or execution of said instructions by said processing means causing the implementation of a method according to the first aim of the invention.

In order to help a practitioner seeking to establish a diagnosis and reach a rapid decision, the communication means of a processing unit according to the invention can deliver an estimated physiological parameter in an appropriate format to output means capable of outputting it to a user.

According to a fourth subject-matter, the invention concerns a Magnetic Resonance imaging analysis system comprising a processing unit according to the invention and output means capable of outputting to a user a physiological parameter according to a method according to the first aim of the invention and implemented by the said processing unit.

Lastly, according to a fifth subject-matter, the invention concerns a computer program product comprising one or more instructions that can be interpreted or executed by the processing means of a processing unit according to the invention. Said processing unit also comprises means for storage or cooperating with such storage means, said program being loadable into said storage means. The said instructions by said processing means are such that their interpretation or execution causes the implementation of a method according to the first subject-matter of the invention.

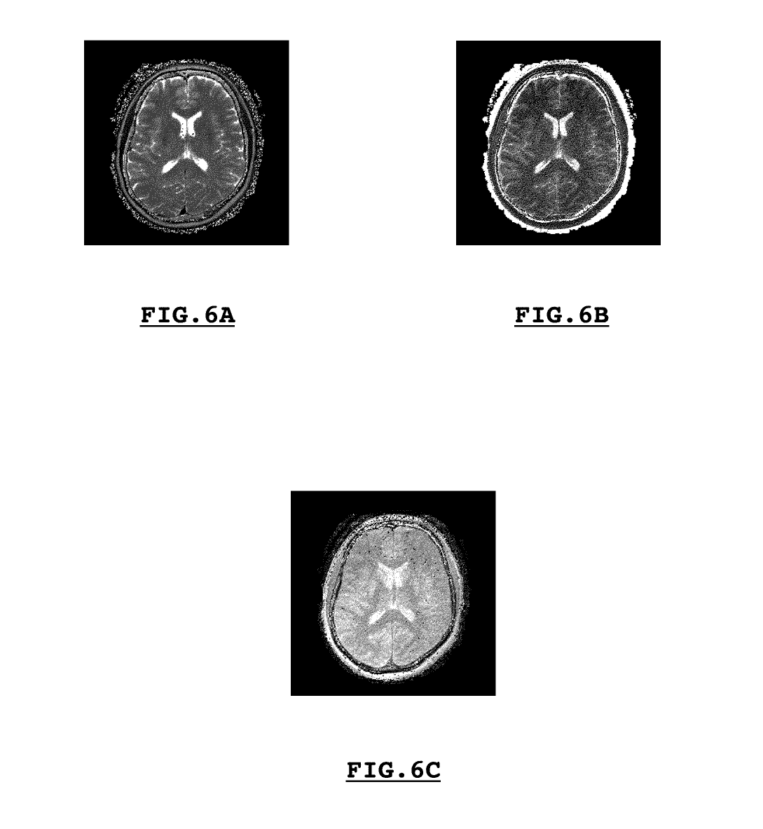

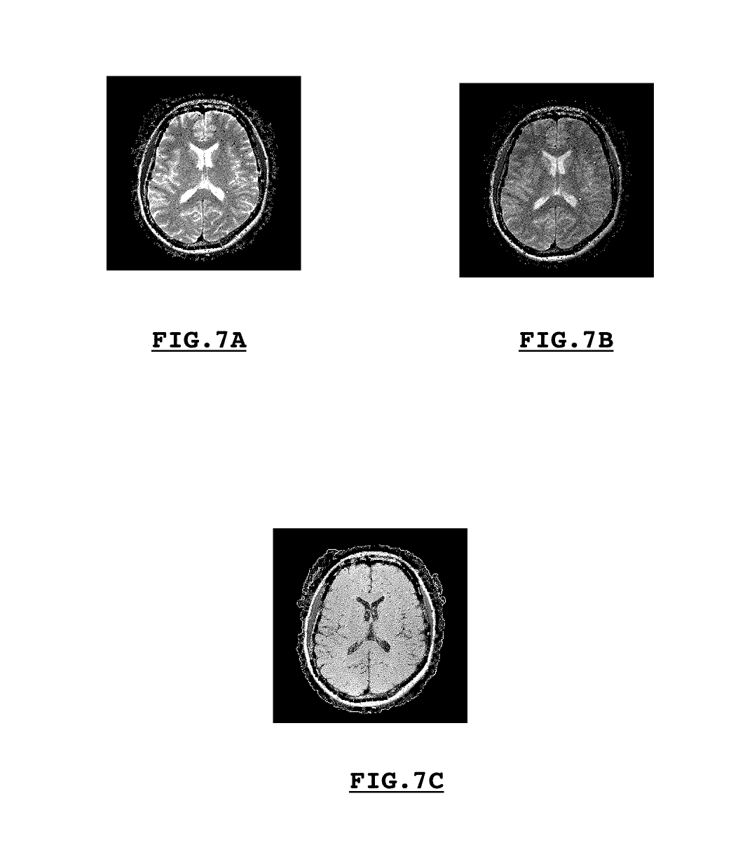

Further features and advantages will emerge more clearly from the following description and the examination of the accompanying Figures, in which:
FIGS. 1 and 2, previously described, show two variations of a medical imaging analysis system, possibly by Magnetic Resonance;
FIGS. 3A, 3B and 3C, previously described, show three examples of weighted maps or images obtained by a Nuclear Magnetic Resonance Imaging device according to the state of the art;
FIG. 4, previously described, shows a simplified flow chart of a method according to the state of the art;
FIG. 5 is a schematic representation of a simplified flow chart of a method according to the invention;
FIGS. 6A, 6B and 6C show three examples of maps of physiological parameters, estimated according to a method according to the invention;
FIGS. 7A, 7B and 7C show three examples of weighted images generated and output according to a method according to the invention.

FIG. 5 is a schematic representation of a method 200 to estimate a physiological parameter of an elementary volume—termed a voxel—of an organ. As a reminder, a “voxel” means any pixel that has a thickness. As previously described, said method 200 is advantageously performed by a processing unit of a Magnetic Resonance Imaging analysis system such as, by way of non-limiting examples, those described in relation to FIGS. 1 and 2. A method 200 according to the invention advantageously comprises a step 230 for estimating said physiological parameter.

An example of implementation of said method 200 will in an advantageous but non-limiting way be described below.

A method 200 according to the invention also comprises a step 221 for estimating a first physiological parameter on the basis of first experimental signals resulting from a first acquisition of signals. Furthermore, the method also involves a step 222 for estimating a second physiological parameter on the basis of second experimental signals resulting from a second acquisition of signals. As previously described, such first and second experimental signals can be directly downloaded from a server, advantageously arranged to store said first and second signals.

Alternatively or additionally, the method 200 according to the invention can comprise steps 211, 212 in order to produce first and second experimental signals respectively on the basis of first and second acquisitions of signals. Thus, such a step 211 can advantageously consist in implementing a first acquisition of signals on the basis of a first acquisition sequence determined in order to estimate a first physiological parameter. Similarly, a step 212 can advantageously consist in implementing a second acquisition of signals on the basis of a second acquisition sequence determined in order to estimate a second physiological parameter. The selection of the first and/or second acquisition sequences can be performed automatically or manually, during a prior step of configuration of the implementation of a method 200 according to the invention, for example via the parameters 16 described previously in relation to FIGS. 1 and 2.

According to a preferred but non-limiting embodiment of the invention, said first and second physiological parameters to be estimated can be the physiological parameters T1 or T2. Furthermore, according to said embodiment, preferably but in a non-limiting way, the sequences of the first and second acquisitions of signals can advantageously consist in any two respective type T1 and T2 mapping sequences, or indeed T2* mapping sequences in the case of a gradient echo acquisition sequence.

Consequently, by way of a non-limiting example, said step 211 to produce the first experimental signals on the basis of a first acquisition of signals can comprise the use of a gradient echo sequence with different flip angles a to estimate the first physiological parameter T1. Such a sequence is particularly advantageous because it is very fast and available for any type of Magnetic Resonance Imaging analysis system. Thus, step 221 for estimating the first physiological parameter can consist in a step of calculation by linearization of an equation linking the said first physiological parameter to the first experimental signals S. As a variation, in a preferred but non-limiting way, step 221 to estimate the first physiological parameter T1 can consist in a step of estimation by a Bayesian method. By way of a non-limiting example, said Bayesian method is described in document WO2012049421 filed by the OLEA MEDICAL Company or also in document WO2010139895A1 also filed by the OLEA MEDICAL Company. Said Bayesian methods can consist in the estimation of the first physiological parameter by calculating its posterior marginalized distribution. Thus, such Bayesian methods increase in particular the accuracy of the estimations and reduce sensitivity to noise.

Similarly, by way of a non-limiting example, step 212 to produce second experimental signals on the basis of a second acquisition of signals can comprise the use of a spin echo sequence at various echo values in order to estimate the second physiological parameter T2. As with the first signal acquisition sequence, such a second sequence is particularly advantageous since it is very fast and available for any Magnetic Resonance Imaging analysis system whatsoever. Furthermore, step 222 for estimating the second physiological parameter can consist in a step of calculation by linearization of an equation linking said second physiological parameter to the second experimental signals S. As a variation, in a preferred but non-limiting way, step 222 for estimating the second physiological parameter T2 may consist in a step of estimation by a Bayesian method. Said Bayesian methods can consist in estimating the second physiological parameter by calculating its marginalized posterior distribution. Such a Bayesian method increases in particular the accuracy of the estimations and reduces sensitivity to noise. Thus, the second acquisition sequence can then be shorter in time, providing results that are qualitatively identical to those obtained with a longer sequence. As a variation, thanks to using a Bayesian method, the spatial resolution of the images, and so the noise level, can be significantly improved without degrading the estimations.

Furthermore, step 230 for estimating the physiological parameter of a method 200 according to the invention involves producing the estimated value of said physiological parameter on the basis of respective estimations of the first and second physiological parameters. In a preferred but non-limiting way, when said first and second estimated physiological parameters are physiological parameters T1 or T2 respectively, the physiological parameter to be estimated can be the physiological parameter PD.

As described in relation to FIGS. 1 and 2, the magnetic Resonance Imaging analysis system can comprise means of output 5 to a user 6, said output means 5 cooperating advantageously with the processing unit 4. Such output means enable an advantageously graphic, sound or other rendering to be provided and can comprise, for example, a screen or loudspeakers. In this case, a method 200 according to the invention can also comprise a subsequent step for triggering an output of the estimated physiological parameters and/or of the first and second physiological parameters in an appropriate format. According to the preferred example of application in which the first and second physiological parameters are the physiological parameters T1 and T2 respectively, the estimated physiological parameter is the physiological parameter PD, such a output can consist in a graphic representation in the form of maps of the first and second physiological parameters T1 and T2 and/or the physiological parameter PD or even one or more estimated values of the first and second physiological parameters T1 and T2 and/or the physiological parameter PD.

Furthermore, in an advantageous but non-limiting way, a method 200 according to the invention can also comprise one or more steps of pre-processing the first and/or second experimental signals obtained respectively on the basis of the first and/or second acquisitions of signals by Magnetic Resonance, the step or steps consisting in correcting said first and/or second experimental signals, in particular by artifact correction or the application of any other corrective filter. By way of non-limiting examples, such steps can consist in the steps of:
correction of movement if the patient does not keep sufficiently still during the acquisition of a sequence. For example, a rigid or non-rigid registration algorithm can be chosen;
co-registration or recalibration between the first and second acquisition sequences if the field of view of said sequences is changed, or if the patient has moved between the first and second sequences. Such a co-registration can advantageously take the form of a rigid or non-rigid co-registration algorithm;
a step of noise reduction in the acquisitions of the two sequences. For example, such a noise reduction step can advantageously take the form of an image convolution smoothing algorithm with a Gaussian kernel;
a step of correcting inhomogeneities of B1 magnetic fields applied within the Magnetic Resonance Imaging device that commonly affect Magnetic Resonance experimental signals.

The invention also concerns a method 300 for producing an estimation of a physiological parameter of a region of interest. A “region of interest” means any region containing at least one voxel. However, a region of interest need not be limited to one voxel, but can comprise a plurality of voxels, advantageously selected manually or automatically. According to the invention, said physiological parameter can be estimated for each voxel by means of a method 200 according to the invention, such as previously described, in particular in relation to FIG. 5, said method being implemented iteratively for each voxel by the processing means of the processing unit 4.

As with a method 200 to estimate a physiological parameter of a voxel, a method 300 according to the invention can also comprise a subsequent step 350 to trigger an output of the estimated physiological parameter and/or of the first, second physiological parameters in an appropriate format, when the Magnetic Resonance Imaging analysis system comprises means 5 of output to a user 6, said output means 5 cooperating advantageously with the processing unit 4. According to a preferred example of application in which the first and second physiological parameters are respectively the physiological parameters T1 and T2, the physiological parameter PD, such an output can consist in the display or printing of a graphic representation in the form of maps of the first, second physiological parameter T1 1 and T2 and/or the physiological parameter PD or even one or several estimated values of the first, second physiological parameters T1 and T2 and/or the physiological parameter PD. Examples of such parameter maps will be described later in relation to FIGS. 6A, 6B and 6C.

Alternatively or additionally, a method 300 according to the invention to estimate a physiological parameter of a region of interest can also comprise a subsequent step 340 to generate a weighted image on the basis of the values produced from an estimated physiological parameter, the first and second physiological parameters for a predetermined acquisition sequence, when the magnetic Resonance Imaging analysis system comprises output means 5 of said system, said output means cooperating with the processing unit 4. Such step 340 enables, in particular, valuable information to be obtained concerning the physiological parameters and one or more weighted images to be generated on the basis of any type of chosen acquisition sequence whatsoever, without requiring the performance of a new examination, and consequently a new acquisition, which is extremely costly in terms of time and money. By way of non-limiting examples, the first, second physiological parameters and the estimated physiological parameter can be, in an advantageous and non-limiting way, the physiological parameters T1, T2 and PD respectively. Advantageously, the method 300 can comprise a configuration step (not shown in FIG. 5), prior to step 340 to generate a weighted image, to select an acquisition sequence and the associated acquisition parameters, such as, by way of non-limiting examples, parameters TR, TE and TI. Such a selection of sequences and parameters can be performed manually by a user or even be implemented automatically. Examples of such weighted images will be further described in relation to FIGS. 7A, 7B and 7C.

Alternatively or additionally, a method 300 according to the invention can advantageously comprise a step (not shown in FIG. 5) to selectively segment a tissue on the basis of known theoretical values of said tissue. For example, let us suppose that the values of T1 and T2 of white matter are known and are worth 560±30 ms and 77±5 ms respectively. A segmentation based on estimated threshold values of T1 and T2 enables the voxels of white matter to be extracted according to the following equation:

whitematter={voxeli|T₁ᵢ ε[530 ms; 590 ms]∪T₂ᵢ ε[72 ms; 82 ms]}

Alternatively or additionally, another example of use of said weighted images for segmentation purposes would consist in using the estimated values of T1, T2 and PD as input data of a partitioning algorithm like the k-means algorithm.

We will now describe an example of implementation of a method 200 according to the invention, an example of which is described in FIG. 5, in order to estimate respectively the physiological parameters T1, T2 and PD and then generate a weighted image or map on the basis of the estimations of the physiological parameters T1, T2 and PD for a predetermined acquisition sequence of the spin echo or inversion-recovery type.

We will first describe steps 212 and 222 of such a method 200 in order to estimate the second physiological parameter T2. As stated above, step 212 can advantageously consist in implementing a second acquisition of signals on the basis of a second acquisition sequence determined in order to estimate a second physiological parameter. Furthermore, as previously described, such a second acquisition sequence can advantageously be a T2 mapping sequence. In a preferred but non-limiting way, said T2 mapping sequence, implemented by processing means of a processing unit 4 of a Magnetic Resonance Imaging analysis system, can advantageously be a multi-echo spin echo sequence. When using such a multi-echo spin echo sequence, the experimental signal in each voxel can be calculated thanks to a decreasing exponential function such as:

S(TE)=S₀e^{TE/T2}

With: S₀ ∝ PD[1−e^{-TR/T1}]

In principle, step 222 to estimate the second physiological parameter T2 consists in a sub-step of calculation by linearization of the preceding equation by taking the logarithm of the experimental signal combined with a sub-step of linear regression. However, the use of such sub-steps is not satisfactory as said sub-steps have high calculation uncertainties.

In a preferable but non-limiting way, step 222 to estimate the second physiological parameter T2 can consist in a Bayesian estimation, such as that described, as stated previously, in document WO2012049421 or even that described in document WO2010139895A1.

In principle, a model is predefined manually or automatically. Bayes' theorem can then be applied, producing an equation linking the posterior distribution of parameters P(T₂, S₀, σ|D) of said predefined model to the prior distributions of said parameters P(T₂), P(S₀), P(σ) and to the likelihood function P(D|T₂, S₀, σ), the likelihood function being defined as the probability distribution of the data knowing the parameters, such as:

P(T₂, S₀, σ|D)∝ P(D|T₂, S₀, σ)·P(T₂)·P(S₀)·P(σ)

where σ is the standard deviation of the noise affecting the data D in a voxel of interest. In our context of application, the data D correspond to the second experimental signals obtained by the acquisition of a second sequence. Conventionally, the estimation of any parameter of interest is performed with the aid of the marginalized posterior distribution estimation of said parameter of interest.

For example, the estimation of the marginalized posterior distribution of the second physiological parameter T2 can be calculated for a voxel of interest by the evaluation of the relation:

P(T₂|D)∝ ∫∫ P(T₂, S₀, σ|D)dS₀ dσ

Then, by way of non-limiting examples, an estimation of the second physiological parameter T2 can finally be calculated in the form of the posterior maximum

[symbol]

T₂ [symbol] =arg max P(T₂|D)

or even the average of the posterior distribution

$$〈 {T}_{2} 〉 = \frac{\int {T}_{2} \cdot P ( {T}_{2} \vert D ) {dT}_{2}}{\int P ( {T}_{2} \vert D ) {dT}_{2}}$$

Said calculations are advantageously implemented by the processing means of a processing unit 4 of a Magnetic Resonance Imaging analysis system according to the invention.

Before this, in order to be able to estimate the marginalized posterior distribution of the second physiological parameter T2, the method comprises sub-steps to calculate, estimate and/or select the prior distributions of these parameters P(T₂), P(S₀), P(σ) and the likelihood function P(D|T₂, S₀, σ). In the absence of additional information about noise, by applying the Maximum Entropy theorem, a Gaussian distribution can be chosen to enable calculation of the likelihood function. Such a choice can be made automatically or manually, at a prior configuration step of implementing a method 200 according to the invention, for example via parameters 16 described above in relation to FIGS. 1 and 2. The likelihood function in 222 is thus calculated:

$$P ( D \vert {T}_{2} , {S}_{0} , σ ) ∝ {σ}^{- N} {e}^{- \frac{{\sum }_{i = 1}^{N} {[ S ( {TE}_{i} ) - {S}_{0} {e}^{\frac{{TE}_{i}}{{T}_{2}}} ]}^{2}}{2 {σ}^{2}}}$$

where N is the number of echo times used to achieve the acquisition.

As for the prior distributions of the parameters, these can be chosen manually or automatically, also during a prior configuration step of implementing a method 200 according to the invention, for example via the parameters 16 described above in relation to FIGS. 1 and 2, such as, by way of non-limiting examples:

P(T₂)∝T₂⁻¹

P(S₀)∝1

P(σ)∝σ⁻¹

Once the prior distributions of said parameters P(T₂), P(S₀), P(σ) of the model and the likelihood function P(D|T₂, S₀, σ) respectively are selected and/or chosen, the marginalized posterior distribution of the second physiological parameter T2 for a given voxel can then be produced, such as:

$$P ( {T}_{2} \vert D ) ∝ {\frac{1}{\sqrt{\sum [ {e}^{- 2 \frac{TE}{{T}_{2}}} ]}} [ 1 - \frac{{( \sum [ {e}^{- \frac{TE}{{T}_{2}}} S ( TE ) ] )}^{2}}{( \sum [ {e}^{- 2 \frac{TE}{{T}_{2}}} ] ) \cdot ( \sum {S ( TE )}^{2} )} ]}^{- \frac{N - 1}{2}}$$

where the sums are made on the different echo times of the acquisition TE. On the basis of this posterior distribution, an estimation of the second physiological parameter T2 of the voxel of interest can be calculated.

Lastly, the parameters S₀ and σ at the voxel of interest can be produced analytically as:

$$〈 {S}_{0} 〉 = \frac{\sum [ {e}^{- \frac{TE}{{T}_{2}}} S ( TE ) ]}{\sum [ {e}^{- 2 \frac{TE}{{T}_{2}}} ]}$$

$$〈 {σ}^{2} 〉 = \frac{1}{N - 3} [ \sum {S ( TE )}^{2} ] \cdot [ 1 - \frac{{( \sum {e}^{- \frac{TE}{{T}_{2}}} S ( TE ) )}^{2}}{( \sum {e}^{- 2 \frac{TE}{{T}_{2}}} ) \cdot ( \sum {S ( TE )}^{2} )} ]$$

Thanks to this analytical calculation, the estimation of parameters S₀ and T2 is then optimal and much less sensitive to the measurement noise than the methods conventionally used.

FIG. 6A shows a map for estimating the second physiological parameter T2, resulting from an iterative implementation of method 200 for a plurality of voxels.

Secondly, we shall describe steps 211 and 221 of a method according to the invention for estimating the first physiological parameter T1. Several acquisition sequences can be used to estimate the first physiological parameter T1. As stated above, step 211 can advantageously consist in implementing a first acquisition of signals on the basis of a first acquisition sequence determined to estimate a first physiological parameter. Furthermore, as previously described, said first acquisition sequence can advantageously be a T1 mapping sequence. By way of non-limiting examples, said T1 mapping sequence, implemented by processing means of a processing unit 4 of a Magnetic Resonance Imaging analysis system, can advantageously be an inversion-recuperation sequence, a look-locker sequence or even a variable flip angle sequence. In a preferable but non-limiting way, a variable flip angle (VFA) sequence can also be used. This sequence is in fact the quickest sequence compared to the previous ones. When using said variable flip angle sequence, the experimental signal in each voxel of interest can be expressed in the form of a proportionality relation, such as:

$$S ∝ {M}_{0} \frac{sin α [ 1 - {e}^{- TR / {T}_{1}} ]}{1 - cos α \cdot {e}^{- TR / {T}_{1}}}$$

$$and$$

$${M}_{0} ∝ {PDe}^{- {T}_{2}^{*} / TE}$$

A conventional estimation of parameters T1 and M₀ consists in performing a linearization calculation of the above proportionality relation. On noting

$${E}_{10} = {e}^{- \frac{TR}{{T}_{1}}} ,$$

the step consisting in calculating the following proportionality relation can then be implemented:

$$\frac{S ( α )}{sin α} = {E}_{10} \frac{S ( α )}{tan α} + {M}_{0} \cdot ( 1 - {E}_{10} )$$

Such an equation can be solved by the least squares method, in order quickly to estimate T1 and M₀ in each voxel. However, this method of estimating the first physiological parameter T1 is very sensitive to noise. Consequently, in a preferable but non-limiting way, step 221 to estimate the first physiological parameter T1 can advantageously consist in a Bayesian estimation, such as that described, as stated previously, in document WO2012049421 or even that described in document WO2010139895A1.

In principle, a model is predefined manually or automatically. Bayes' theorem can then be applied, producing an equation linking the posterior distribution of parameters P(T₁, M₀, σ|D) of said predefined model to the prior distributions of said parameters P(T₁), P(M₀), P(σ) and to the likelihood function P(D|T₁, M₀, σ), the likelihood function being defined as the probability distribution of the data knowing the parameters, such as:

P(T₁, M₀, σ|D)∝ P(D|T₁, M₀, σ)·P(T₁)·P(M₀)·P(σ)

where σ is the standard deviation of the noise affecting the data D in a voxel of interest. In our context of application, the data D correspond to the first experimental signals obtained by the acquisition of the said first sequence. Conventionally, as previously described, the estimation of any parameter of interest is performed with the aid of the marginalized posterior distribution estimation of said parameter of interest.

The estimation of the marginalized posterior distribution of the first physiological parameter T1 can advantageously be calculated for a voxel of interest as:

P(T₁|D)∝ ∫∫ P(T₁, M₀, σ|D)dM₀ dσ

Then, by way of non-limiting examples, an estimation of the first physiological parameter T1 can be calculated for example in the form of the posterior maximum

[symbol]

T₁ [symbol] =arg max P(T₁|D)

or even the average of the posterior distribution

$$〈 {T}_{1} 〉 = \frac{\int {T}_{1} \cdot P ( {T}_{1} \vert D ) {dT}_{1}}{\int P ( {T}_{1} \vert D ) {dT}_{1}}$$

Said calculations are advantageously implemented by the processing means of a processing unit 4 of a Magnetic Resonance Imaging analysis system according to the invention.

Before this, in order to be able to estimate the marginalized posterior distribution of the first physiological parameter T1, the method, more particularly step 221, advantageously comprises sub-steps to calculate, estimate and/or select the prior distributions of these parameters P(T₁), P(M₀), P(σ) and the likelihood function P(D|T₁, M₀, σ). In the absence of additional information about noise, by applying the Maximum Entropy theorem, a Gaussian distribution can be chosen to enable calculation of the likelihood function. Such a choice can be made automatically or manually, at a prior configuration step of implementing a method 200 according to the invention, for example via parameters 16 described above in relation to FIGS. 1 and 2. The likelihood function in 221 is thus calculated:

$$P ( D \vert {T}_{1} , {M}_{0} , σ ) ∝ {σ}^{- N} {e}^{- \frac{{\sum }_{i = 1}^{N} {[ S ( {α}_{i} ) - {M}_{0} \frac{sin α [ 1 - {e}^{- \frac{TR}{{T}_{1}}} ]}{1 - cos α \cdot {e}^{- \frac{TR}{{T}_{1}}}} ]}^{2}}{2 {σ}^{2}}}$$

where N is the number of flip angles used to achieve the acquisition. As for the prior distributions of the parameters, these can be chosen manually or automatically, also during a prior configuration step of implementing a method 200 according to the invention, for example via the parameters 16 described above in relation to FIGS. 1 and 2, such as, by way of non-limiting examples:

P(T₁)∝T₁¹

P(M₀)∝1

P(σ)∝σ⁻¹

While noting:

$$f ( α , {T}_{1} ) = \frac{sin α [ 1 - {e}^{- \frac{TR}{{T}_{1}}} ]}{1 - cos α \cdot {e}^{- \frac{TR}{{T}_{1}}}}$$

Once the prior distributions of said parameters P(T₁), P(M₀), P(σ) and the likelihood function P(D|T₁, M₀, σ) respectively are selected and/or chosen, the marginalized posterior distribution of the first physiological parameter T1 for a given voxel can then be produced, such as:

$$P ( {T}_{1} \vert D ) ∝ {\frac{1}{\sqrt{\sum [ {f ( α , {T}_{1} )}^{2} ]}} [ 1 - \frac{{( \sum [ f ( α , {T}_{1} ) S ( α ) ] )}^{2}}{( \sum [ {f ( α , {T}_{1} )}^{2} ] ) \cdot ( \sum {S ( α )}^{2} )} ]}^{- \frac{N - 1}{2}}$$

where the sums are made on the different flip angles of acquisition T1. On the basis of this posterior distribution, an estimation of the first physiological parameter T1 of the voxel of interest can be calculated.

Lastly, the parameters M₀ and σ at the voxel of interest can be calculated analytically as:

$$〈 {σ}^{2} 〉 = \frac{1}{N - 3} [ \sum {S ( α )}^{2} ] \cdot [ 1 - \frac{{( \sum [ f ( α , {T}_{1} ) S ( α ) ] )}^{2}}{( \sum [ {f ( α , T 1 )}^{2} ] ) \cdot ( \sum {S ( α )}^{2} )} ]$$

Thanks to this analytical calculation, the estimation of parameters M₀ and T1 is then optimal and much less sensitive to the measurement noise than the methods conventionally used.

FIG. 6B shows a map for estimating the first physiological parameter T1, resulting from an iterative implementation of method 200 for a plurality of voxels.

Thirdly, we shall describe step 230 for estimating the physiological parameter PD.

In the context of our example, parameter S₀ has been estimated thanks to step 222 for estimating the second physiological parameter T2 and to the T2 mapping sequence. Said parameter S₀ depends on the physiological parameter PD, and is T1 weighted. The parameter S₀ can thus be calculated according to the following proportionality relation, such as:

$${S}_{0} ∝ PD [ 1 - {e}^{- \frac{TR}{{T}_{1}}} ]$$

By having an estimation of S₀, but also of the first physiological parameter T1 thanks to the T1 mapping sequence, the physiological parameter PD can be estimated in 230 at the voxel concerned as:

$$〈 PD 〉 ∝ \frac{〈 {S}_{0} 〉}{1 - {e}^{- \frac{TR}{〈 {T}_{1} 〉}}}$$

where the acquisition parameter TR corresponds to the repetition time of the T2 mapping sequence.

Said calculations, used in step 230 to estimate the physiological parameter PD, are advantageously implemented by the processing means of a processing unit 4 of a Magnetic Resonance Imaging analysis system according to the invention. Such an estimation of the physiological parameter PD is relative and proportional to the real value of the estimated physiological parameter PD. However, the proportionality factor between the estimation and the real value of PD depends solely on the properties of the Magnetic Resonance Imaging device. Thus, such uncertainty, in the form of a relative value, poses no problem to the generation of a synthetic weighted image or MRI map.

FIG. 6C shows an estimation map of the physiological parameter PD, resulting from an iterative implementation of method 200 for a plurality of voxels.

Lastly, we will describe the step for generating a weighted image on the basis of the estimations of the first, second estimated physiological parameters T1, T2 and PD respectively for a particular acquisition sequence. According to the invention, “weighted image” means any T1, T2 weighted image or any inversion-recovery image: the invention will not be limited to the term “weighted”.

By cleverly combining the acquisition parameters of a Nuclear Magnetic Resonance Imaging device and the previously estimated physiological parameters, a user of said device can order the device to generate images, maps or sequences of T1, T2, PD weighted images, or even conceal and/or mask certain types of tissues on the basis of a chosen acquisition sequence, as well as the associated acquisition parameters. As a variation, the invention envisages that such map images or sequences of T1, T2, PD weighted images can be generated automatically and output by a Magnetic Resonance Imaging analysis system advantageously comprising a processing unit 4 and output means 5 advantageously cooperating with said processing unit 4. The acquisition sequence as well as the associated acquisition parameters can thus be chosen automatically.

By way of non-limiting examples, we would mention here, in an advantageous but non-limiting way, several sequences that the Magnetic Resonance Imaging analysis system can calculate.

According to a first embodiment, for a so-called “conventional” spin echo acquisition sequence, the method for estimating and generating may comprise a step for calculating in each voxel i:

Sᵢ=PDᵢ(1−e^{-TR/T}^{₁ᵢ})e^{-TE/T}^{₂ᵢ}

where PDᵢ, T₁ᵢ and T₂ᵢ are the estimations of the physiological parameters T1, T2, PD at the voxel i previously produced for each voxel i. On the basis of this sequence, the Magnetic Resonance Imaging analysis system can generate and output, by means of its processing unit and its output means, T1 or T2 weighted synthetic images.

In the same way, as a variation, according to a second embodiment, for an inversion-recovery type acquisition sequence, the method for estimating and generating may include a step for calculating in each voxel i:

$${S}_{i} = {PD}_{i} \cdot ( 1 - 2 {e}^{- \frac{TI}{{T}_{1 i}}} + {e}^{- \frac{TR}{{T}_{1 i}}} ) {e}^{- \frac{TE}{{T}_{2 i}}}$$

The method can then comprise a step for generating an inversion-recovery image. This type of sequence allows certain types of tissues, such as liquids, to be suppressed.

As a variation, according to a third embodiment, for a saturation-recovery type acquisition sequence, the method for estimating and generating can include a step for calculating in each voxel i:

$${S}_{i} = {PD}_{i} \cdot ( 1 - {e}^{- \frac{TR}{{T}_{1 i}}} )$$

FIGS. 7A, 7B and 7C show three examples of weighted images generated according to the method according to the invention. Respectively, said FIGS. 7A, 7B and 7C show respectively T2 weighted, T1 weighted and inversion-recovery synthetic images, said inversion-recovery image highlighting the suppression of water. FIG. 7A shows a T2 weighted image based on a spin echo sequence chosen with an echo time defined at one hundred and twenty milliseconds and a repetition time defined at one thousand five hundred milliseconds. Similarly, FIG. 7B shows a T1 weighted image based on a spin echo sequence chosen with an echo time defined at thirty milliseconds and a repetition time defined at five thousand milliseconds. Lastly, FIG. 7C shows a weighted image based on an inversion-recovery sequence chosen with an echo time defined at fifty milliseconds, a repetition time defined at twenty thousand milliseconds and an inversion time defined at one thousand seven hundred milliseconds.

Thanks to the new estimations and/or maps described above, the invention provides a practitioner with an entire set of relevant and consistent data that is quickly available thanks to the use of a method according to the invention. This availability is made possible by an adaptation of the processing unit 4 according to FIG. 1 or 2, in that the processing means implement a method of estimating a physiological parameter of a voxel or a region of interest comprising the production of the estimated value of said physiological parameter from respective estimations of the first and second physiological parameters. Such implementation is advantageously made possible by downloading or recording, within the storage means cooperating with said processing means, of a computer program product. The latter in fact comprises instructions that can be interpreted and/or executed by said processing means. The interpretation or execution of said instructions triggers the implementation of a method 200 or 300 according to the invention. The means for communicating with the outside world of said processing unit can deliver a physiological parameter, namely the estimated parameters 14, in an appropriate format to output means capable of outputting it to a user 6, said estimated physiological parameter being advantageously outputtable in the form, for example, of weighted maps or images such as those illustrated in FIGS. 6A to 6C and 7A to 7C. Thanks to the invention, the data provided are more numerous, consistent, reproducible and accurate. The data, made available to the practitioner, are thus of a sort to increase the practitioner's confidence and speed in determining his diagnosis and reaching a decision.

1. Method for producing an estimation of a physiological parameter of an elementary volume—termed a voxel—of an organ, said method being implemented by processing means of a processing unit of a Magnetic Resonance Imaging analysis system, and comprising a step for estimating said physiological parameter, said method further comprising:
a step for estimating a first physiological parameter on the basis of first experimental signals resulting from a first acquisition of signals, and
a step for estimating a second physiological parameter on the basis of second experimental signals resulting from a second acquisition of signals,
wherein said step for estimating the physiological parameter comprises producing the estimated value of said physiological parameter on the basis of respective estimations of the first and second physiological parameters.

2. Method according to claim 1, also comprising steps to produce the first and second experimental signals respectively on the basis of first and second acquisitions of signals.

3. Method according to claim 1, wherein the step for estimating a first physiological parameter comprises a step of estimation by a Bayesian method, including estimating said first physiological parameter by calculating its marginalized posterior distribution.

4. Method according to claim 1, wherein the step for estimating a second physiological parameter comprises a step of estimation by a Bayesian method, including estimating said second physiological parameter by calculating its marginalized posterior distribution.

5. Method according to claim 1, wherein the Magnetic Resonance Imaging analysis system comprises means for outputting the estimated parameter to a user of said system, said output means cooperating with the processing unit, said method comprising a subsequent step for triggering an output of the estimated physiological parameter and/or of the first and second physiological parameters.

6. Method according to claim 1, also comprising a prior step of pre-processing the first and/or second experimental signals obtained from the first and/or second acquisitions by Magnetic Resonance respectively, said step being arranged to correct the said first and/or second experimental signals.

7. Method for producing an estimation of a physiological parameter of a region of interest, said region comprising at least one voxel, said physiological parameter being estimated for each voxel by means of a method according to claim 1.

8. Method according to claim 7, when the Magnetic Resonance Imaging analysis system comprises means of output to a user of said system, said output means cooperating with the processing unit, also comprising a subsequent step for triggering the output of the estimated physiological parameter, the first and/or second physiological parameters for each voxel of the region of interest in the form of a map describing a physiological parameter.

9. Method according to claim 7, when the Magnetic Resonance Imaging analysis system comprises means of output to a user of said system, said output means cooperating with the processing unit, also comprising a subsequent step to generate a weighted image on the basis of the values produced from the estimated physiological parameter, the first and second physiological parameters for a predetermined acquisition sequence.

10. Processing unit comprising:
means to communicate with the outside world and processing means, cooperating with storage means, wherein:
the communication means are arranged to receive from the outside world signals based on the first and/or second acquisitions of signals by Magnetic Resonance;
the storage means contain instructions that can be executed or interpreted by the processing means, the interpretation or execution of said instructions by said processing means causing the implementation of a method according to claim 1.

11. Processing unit according to claim 10, wherein the communication means deliver an estimated physiological parameter in an appropriate format to output means capable of outputting it to a user.

12. Magnetic Resonance Imaging analysis system comprising a processing unit having means to communicate with the outside world and processing means, cooperating with storage means, wherein the communication means are arranged to receive from the outside world signals based on the first and/or second acquisitions of signals by Magnetic Resonance, and the storage means contain instructions that can be executed or interpreted by the processing means, wherein the interpretation or execution of said instructions by said processing means causes the implementation of a method according to claim 1, and output means capable of outputting to a user a physiological parameter according to the method implemented by said processing unit.

13. A non-transitory computer-readable medium encoded with a program comprising one or more instructions that can be interpreted or executed by the processing means of a processing unit, said processing unit also comprising means for storage or cooperating with such storage means, said program being loadable into said storage means, wherein the interpretation or execution of said instructions by said processing means causes the implementation of a method according to claim 1.
